(19) United States
(12) Patent Application Publication
(10) Pub. No.: US 2023/0285288 A1
(43) Pub. Date: Sep. 14, 2023

(54) NANOPARTICLE COMPOSITIONS COMPRISING CANNABINOIDS

(71) Applicant: Miguel Berkstrom, Comptche, CA (US)

(72) Inventor: Miguel Berkstrom, Comptche, CA (US)

(21) Appl. No.: 18/106,659

(22) Filed: Feb. 7, 2023

Related U.S. Application Data
(63) Continuation of application No. 16/713,154, filed on Dec. 13, 2019, now abandoned.
Application No. 16/713,154 is a continuation of application No. 15/944,647, filed on Apr. 3, 2018, now abandoned.
(60) Provisional application No. 62/481,555, filed on Apr. 4, 2017.
(60) Provisional application No. 62/558,132, filed on Sep. 13, 2017.

(51) Int. Cl. A61K 9/107; A61K 47/26; A61P 29/00; A61K 31/05; A61K 36/185; A61K 31/352

(52) U.S. Cl. CPC A61K 9/107; A61K 47/26; A61P 29/00; A61K 31/05; A61K 36/185; A61K 31/352

(57) ABSTRACT

The present disclosure provides a nanoparticulate cannabinoid composition.

### CROSS-REFERENCE TO RELATED APPLICATIONS

This application is a continuation of U.S. patent application Ser. No. 16/713,154 filed on Dec. 13, 2019, which application claims the benefit of U.S. patent application Ser. No. 15/944,647, filed on Apr. 3, 2018, which claims the benefit of the filing date of U.S. Provisional Application No. 62/558,132, filed Sep. 13, 2017, and claims the benefit of U.S. Provisional Application No. 62/481,555, filed Apr. 4, 2017, the disclosures of which are hereby incorporated by reference herein in their entireties.

### BACKGROUND OF THE DISCLOSURE

Cannabinoids are compounds derived from Cannabis sativa, an annual plant in the Cannabaceae family. The plant contains about sixty cannabinoids. The most active naturally occurring cannabinoid is tetrahydrocannabinol (THC), which is used for the treatment of a wide range of medical conditions, including glaucoma, AIDS wasting, neuropathic pain, treatment of spasticity associated with multiple sclerosis, fibromyalgia and chemotherapy-induced nausea. Additionally, THC has been reported to exhibit a therapeutic effect in the treatment of allergies, inflammation, infection, epilepsy, depression, migraine, bipolar disorders, anxiety disorder, and drug dependency and withdrawal syndromes. THC is particularly effective as an anti-emetic drug and is administered to curb emesis, a common side effect accompanying the use of opioid analgesics and anaesthetics, highly active anti-retroviral therapy and cancer chemotherapy.

Cannabinoids are lipophilic and potentially acid-labile compounds. Because of their hydrophobic nature, cannabinoids are poorly absorbed systemically from oral dosage forms because of the poor dissolution of cannabinoids in the aqueous environment of gastrointestinal tract. Oral formulations of cannabinoids, therefore, exhibit low bioavailability.

Oral formulations of synthetic cannabinoids are also available commercially. For instance, Nabilone is a synthetic cannabinoid marketed as Cesamet® in Canada the United States, the United Kingdom and Mexico. Nabilone is formulated as capsules suitable for oral administration. Cesamet® is approved for use as an antiemetic and analgesic for neuropathic pain. Sativex®, is a mouth spray containing tetrahydrocannabinol (THC) and cannabidiol (CBD). It is approved for the treatment of spasticity due to multiple sclerosis. Administration of synthetic cannabinoid formulations show fewer undesirable side effects than THC.

Because of their poor absorption and poor bioavailability, oral formulations have the additional disadvantage that they require several administrations a day, making it inconvenient for patients who have difficulty swallowing.

### BRIEF SUMMARY OF THE DISCLOSURE

In one aspect of the present disclosure is a composition comprising water, an emulsifier, and a cannabinoid, wherein an average particle size of the nanoparticle composition is less than about 750 nm. In some embodiments, the composition is formulated as an emulsion. In some embodiments, the average particle size is less than 500 nm. In some embodiments, an average particle size ranges from between about 50 nm to about 450 nm (see, e.g. FIG. 1). In some embodiments, the average particle size ranges from between about 10 nm to about 200 nm. In some embodiments, the nanoparticle increases the bioavailability of the cannabinoid 2-fold to 10-fold compared to bioavailability without nanoparticle encapsulation. In some embodiments, the nanoparticle increases the bioavailability of the cannabinoid 2-fold to 8-fold compared to bioavailability without nanoparticle encapsulation. In some embodiments, the nanoparticle increases the bioavailability of the cannabinoid 3-fold to 5-fold compared to bioavailability without nanoparticle encapsulation. In some embodiments, the nanoparticle decreases the dose of cannabinoids 2-fold to 10-fold less than an amount of cannabinoids needed to illicit the same therapeutic effect compared to raw and non-encapsulated cannabinoids in a patient in need thereof. In some embodiments, the nanoparticle decreases the dose of cannabinoids 2-fold to 8-fold less than an amount of cannabinoids needed to illicit the same therapeutic effect compared to raw and non-encapsulated cannabinoids in a patient in need thereof In some embodiments, the nanoparticle decreases the dose of cannabinoids 3-fold to 5-fold less than an amount of cannabinoids needed to illicit the same therapeutic effect compared to raw and non-encapsulated cannabinoids in a patient in need thereof. In some embodiments, an amount of cannabinoid is present in the composition ranges from about 0.01% to about 10% by weight of the composition. In some embodiments, the amount ranges from between about 0.1% to about 10%. In some embodiments, an amount of cannabinoid is present in the composition ranges from about 0.01% to about 10% by weight of the composition; and wherein an amount of emulsifier present in the composition ranges from about 0.5% to about 10% by weight of the composition. In some embodiments, the cannabinoid is uniformly distributed through the composition (see FIG. 2).

It is also an object of the present disclosure to provide the composition, as set forth above, wherein the composition is stable at room temperature for about 3 months to about 12 months. It is also an object of the present disclosure to provide the composition as mentioned above, wherein the composition is stable at fridge temperature for about 6 months to about 24 months. It is also an object of the present disclosure to provide the composition as mentioned above, wherein the composition is stable at about 40 degrees Celsius temperature for about 2 months to about 6 months. It is also an object of the present disclosure to provide the composition as mentioned above, wherein stability of the composition is measured using a technique selected from the group consisting of measuring drop size, light scattering, focused beam reflectance measurement, centrifugation, rheology and any combination thereof. The term “stable” refers hereinafter to the stability of the emulsion as disclosed in the present invention, and specifically refers to the ability of the emulsion to resist change in its properties over time. Instability may be manifested in any of the following: flocculation, creaming, coalescence and Ostwald ripening. Determination whether an emulsion has lost its stability may be carried out in any of the following techniques: measurement of particle size, light scattering, focused beam reflectance measurement, centrifugation or rheology.

In another aspect of the present disclosure is a method of treating pain comprising administering to a subject in need thereof a composition comprising water, an emulsifier, and a cannabinoid, wherein an average particle size of the nanoparticle composition is less than about 750 nm, and a pharmaceutically acceptable excipient. In some embodiments, an amount of the composition is administered such that following administration the cannabinoid achieves a concentration ranging from about 0.001 mg/kg to about 100 mg/kg. In some embodiments, an amount of the composition of any of claims 1 to 12 is administered such that following administration the cannabinoid achieves a concentration ranging from about 0.01 mg/kg to about 100 mg/kg. In some embodiments, an amount of the composition of any of claims 1 to 12 is administered such that following administration the cannabinoid achieves a concentration ranging from about 0.1 mg/kg to about 100 mg/kg. In some embodiments, the composition is adapted to be administered in a route selected from a group consisting of: intranasal, transdermal, intravenous, oral, topical, topical and any combination thereof. In some embodiments, the composition used in the aforementioned method may be manufactured to provide emulsion particle sizes in the range of about 50 nm to about 200 nm, or in the range of about 75 nm to about 150 nm. Such particularly small particle size is achieved through using a microfluidizer having a pressure of about 15,000 PSI to about 35,000 PSI.

In another aspect of the present disclosure is a method of creating a temporary euphoric state comprising administering to a subject in need thereof a composition comprising water, an emulsifier, and a cannabinoid, wherein an average particle size of the nanoparticle composition is less than about 750nm, and a pharmaceutically acceptable excipient. In some embodiments, an amount of the composition is administered such that following administration the cannabinoid achieves a concentration ranging from about 0.001 mg/kg to about 100 mg/kg. In some embodiments, an amount of the composition of any of claims 1 to 12 is administered such that following administration the cannabinoid achieves a concentration ranging from about 0.01 mg/kg to about 100 mg/kg. In some embodiments, the composition is administered such that following administration the cannabinoid achieves a concentration ranging from about 0.1 mg/kg to about 100 mg/kg.

In another aspect of the present disclosure is a dietary supplement comprising water, an emulsifier, and a cannabinoid, wherein an average particle size of the nanoparticle composition is less than about 750nm, and a pharmaceutically acceptable excipient. In some embodiments, an amount of the dietary supplement is administered such that following administration the cannabinoid achieves a concentration ranging from about 0.001 mg/kg to about 100 mg/kg. In some embodiments, the composition administered such that following administration the cannabinoid achieves a concentration ranging from about 0.01 mg/kg to about 100 mg/kg. In some embodiments, an amount of the dietary supplement is administered such that following administration the cannabinoid achieves a concentration ranging from about 0.1 mg/kg to about 100 mg/kg.

### BRIEF DESCRIPTION OF THE FIGURES

The patent or application file contains at least one drawing executed in color. Copies of this patent or patent application publication with color drawings will be provided to the Office upon request and the payment of the necessary fee. For a general understanding of the features of the disclosure, reference is made to the drawings. In the drawings, like reference numerals have been used throughout to identify identical elements.

FIG. 1 sets forth various particle size distributions of a composition prepared according to the methods of the present disclosure.

FIG. 2 sets forth data illustrating the uniform distribution of cannabinoids within a composition prepared according to the methods of the present disclosure.

### DETAILED DESCRIPTION

Definitions

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of ” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “administering” means providing a composition, formulation, or specific agent to a subject in need of treatment, including those described herein.

As used herein, the term “subject” refers to a mammal such as a human, mouse or primate. Typically, the mammal is a human (Homo sapiens).

For purposes of the present disclosure, the term “cannabinoid” includes naturally occurring and non-natural derivatives of cannabinoids which can be obtained by derivatization of natural cannabinoids and which are unstable like natural cannabinoids. In other words, the cannabinoid used in the formulations of the present disclosure may be natural, semi-synthetic, or synthetic. The cannabinoid may be included in its free form, or in the form of a salt; an acid addition salt of an ester; an amide; an enantiomer; an isomer; a tautomer; a prodrug; a derivative of an active agent of the present disclosure; different isomeric forms (for example, enantiomers and diastereoisomers), both in pure form and in admixture, including racemic mixtures; enol forms. The term “cannabinoid” is also meant to encompass derivatives that are produced from another compound of similar structure by the replacement of, e.g., substitution of one atom, molecule or group by another such as 11-hydroxy-delta-8-tetrahydrocannabinol and 11-hydroxy-delta-9-tetrahydrocannabinol.

As used herein, the terms “treatment,” “treating,” or “treat,” with respect to a specific condition, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition. The term “treatment” is used in some embodiments to refer to administration of a compound of the present disclosure to mitigate a disease or a disorder in a host, preferably in a mammalian subject, more preferably in humans. Thus, the term “treatment” can include includes: preventing a disorder from occurring in a host, particularly when the host is predisposed to acquiring the disease, but has not yet been diagnosed with the disease; inhibiting the disorder; and/or alleviating or reversing the disorder. Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

Compositions

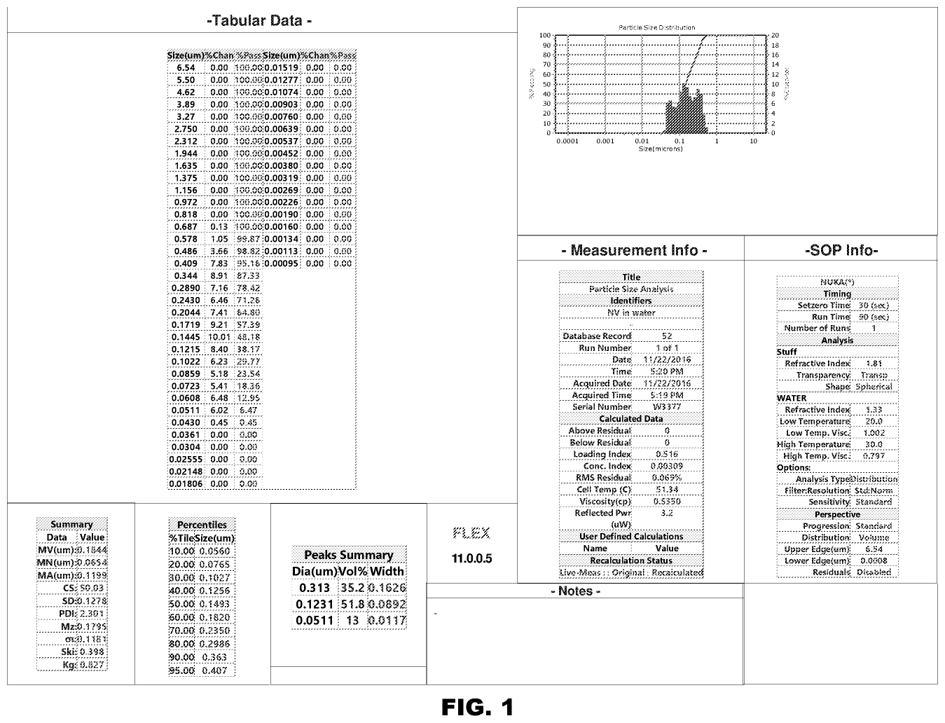

In general, the present disclosure provides for nanoparticle compositions comprising a cannabinoid, an emulsifier, and water, wherein the nanoparticle compositions have an average particle size which is less than 950 nm. In some embodiments, an average particle size is less than about 850 nm. In some embodiments, an average particle size is less than about 750 nm. In some embodiments, an average particle size is less than about 50 nm. In some embodiments, an average particle size ranged from about 10 nm to about 500 nm. In some embodiments, an average particle size ranged from about 10 nm to about 480 nm. In some embodiments, an average particle size ranged from about 10 nm to about 460 nm. In some embodiments, an average particle size ranged from about 10 nm to about 450 nm. In some embodiments, an average particle size ranged from about 10 nm to about 440 nm. In some embodiments, an average particle size ranged from about 10 nm to about 430 nm. In some embodiments, an average particle size ranged from about 10 nm to about 420 nm. In some embodiments, an average particle size ranged from about 10 nm to about 410 nm. In some embodiments, an average particle size ranged from about 10 nm to about 400 nm. In some embodiments, an average particle size ranged from about 10 nm to about 300 nm. In some embodiments, an average particle size ranged from about 10 nm to about 250 nm. In some embodiments, an average particle size ranged from about 10 nm to about 150 nm. In some embodiments, an average particle size ranged from about 50 nm to about 250 nm. In some embodiments, an average particle size ranged from about 50 nm to about 200 nm. In some embodiments, an average particle size ranged from about 25 nm to about 150 nm. In some embodiments, an average particle size ranged from about 25 nm to about 250 nm. In some embodiments, an average particle size ranged from about 25 nm to about 200 nm. In some embodiments, an average particle size ranged from about 10 nm to about 100 nm. In some embodiments, an average particle size ranged from about 10 nm to about 150 nm. In some embodiments, an average particle size ranged from about 5 nm to about 100 nm. In some embodiments, an average particle size ranged from about 5 nm to about 50 nm. In some embodiments, an average particle size ranged from about 50 nm to about 250 nm. In some embodiments, an average particle size ranged from about 50 nm to about 200 nm. Particle size distributions are further illustrated in FIG. 1.

In some embodiments, an average particle size ranged from about 50 nm to about 500 nm. In some embodiments, an average particle size ranged from about 50 nm to about 480 nm. In some embodiments, an average particle size ranged from about 50 nm to about 460 nm. In some embodiments, an average particle size ranged from about 50 nm to about 450 nm. In some embodiments, an average particle size ranged from about 50 nm to about 440 nm. In some embodiments, an average particle size ranged from about 50 nm to about 430 nm. In some embodiments, an average particle size ranged from about 50 nm to about 420 nm. In some embodiments, an average particle size ranged from about 50 nm to about 410 nm.

In some embodiments, an average particle size ranged from about 100 nm to about 500 nm. In some embodiments, an average particle size ranged from about 100 nm to about 480 nm. In some embodiments, an average particle size ranged from about 100 nm to about 460 nm. In some embodiments, an average particle size ranged from about 100 nm to about 450 nm. In some embodiments, an average particle size ranged from about 100 nm to about 440 nm. In some embodiments, an average particle size ranged from about 100 nm to about 430 nm. In some embodiments, an average particle size ranged from about 100 nm to about 420 nm. In some embodiments, an average particle size ranged from about 100 nm to about 410 nm.

Without wishing to be bound by any particular theory, it is believed that the composition is a nanoemulsion in that the cannabinoid particles are emulsified with the surfactant providing repulsive electrostatic interactions and steric hindrance. In some embodiments, the presently disclosed nanoemulsions allow for increased rates of absorption of the cannabinoid and/or a reduction in the variability of absorption of the cannabinoid. In some embodiments, the presently disclosed nanoemulsions allow for increased bioavailability of the cannabinoid.

As used herein, the term “cannabinoid” means any compound that interacts with a cannabinoid receptor. Examples of cannabinoids include, but not limited to, certain tetrahydropyran analogs (Δ9tetrahydrocannabinol, Δ8-tetrahydrocannabinol, 6,6,9-trimythel-3-pentyl-6H-dibenzo[b,d]pyran-1-ol, 3-(1,1-dimethylheptyl)-6,6a7, 8,10,10a-hexahydro-1-1hydroxy-6,6-dimythel-9H-dibezo[b,d]pyran-9-ol,(−)-(3S,4S)-7-hydroxy-delta-6-tetrahydrocannabino1-1,1-dimethylheptyl, (+)-(3S,4S)-7-hydroxy-Δ-6-tetrahydrocannabinol, and 48-tetrahydrocannabinol-11-oic acid); certain piperidine analogs (e.g., (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)); certain aminoalkylindole analogs (e.g., (R)-(+)-[2,3-dihydro-5-methyl-3-(4-morpholinylm-ethyl)-pyrrolo[1,2,3,-de]-1,4-benzoxazin-6-yl]-1-naphthelenyl-methanone); certain open pyran-ring analogs (e.g., 2-[3-methyl-6-(1-methylethenyl-2-cyclohexen-1-yl]-5-pentyl-1, 3-benzendi-ol, and 4-(1,1-dimethylheptyl)-2,3′-dihydroxy-6′-α-(3-hydroxypropyl)-1′,-2′,3′,4′,5′,6′-hexahydrobiphenyl), their salts, solvates, metabolites, and metabolic precursors.

Other examples of cannabinoids include the following: (6aR,10aR)-delta-9-tetrahydrocannabinol (i.e. (−)-(6aR,10aR)-6,6,9-trimethyl- 3 -pentyl-6a,7,8,10a-tetrahydro-6H-benzo[c]chromen-1-ol) (THC); Tetrahydrocannabinolic acid (i.e. (6aR,10aR)-1-hydroxy-6,6,9-trimethyl-3-pentyl-6a,7,8,10a-tetrahydro-6H-benzo[c]chromene-2-carboxylate) (THC-A); Tetrahydrocannabivarin (i.e. 6,6, 9-trimethyl-3-propyl-6a,7,8,10a-tetrahydrobenzo[c]chromen-1-) (THCV); Cannabichromene (i.e. 2-methyl-2-(4-methylpent-3-enyl)-7-pentylchromen-5-ol) (CBC); Cannabigerol (i.e. 2-[(2E)-3,7-dimethylocta-2,6-dienyl]-5-pentylbenzene-1,3 -diol) (CBG); Cannabigerolic acid (i.e. 3-[(2E)-3,7-dimethylocta-2,6-dienyl]-2,4-dihydroxy-6-pentylbenzoic acid) (CBGA); Cannabinol (i.e. 6,6,9-trimethyl-3-pentylbenzo[c]chromen-1-ol) (CNB); Cannabidiol (i.e. 2-[(1R,6R)-3 -methyl-6-prop-1-en-2-ylcyclohex-2-en-1-yl]-5-pentylbenzene-1,3-diol) (CBD); Cannabidivarin (i.e. 2-[(1R,6R)-3 -methyl-6-prop-1-en-2-ylcyclohex-2-en-1-yl]-5-propylbenzene-1,3-diol) (CBDV); and Cannabidiolic Acid (i.e. 2,4-dihydroxy-3-[(1R,6R)-3-methyl-6-prop-1-en-2-ylcyclohex-2-en-1-yl]-6-pentylbenzoic acid) (CBDA).

In addition to natural cannabinoids, the nanoparticle compositions disclosed herein may utilize synthetic cannabinoid compounds as well as cannabinoids and their analogs that are obtained using semi-synthetic protocols. In some embodiments, the manufacture of cannabinoid compounds and their analogs using semi-synthetic means involves contacting an appropriate substrate with one of the cannabinoid synthase enzymes. For example, tetrahydrocannabinolic acid (THCA) or its analogs can be manufactured semi-synthetically by contacting cannabigerolic acid (CBGA) or an appropriately substituted derivative of CBGA with THC synthase to obtain the corresponding THCA or THCA analog respectively. The inventive compositions may also contain natural or synthetically modified cannabinoids.

In some embodiments, cannabinoids may be obtained from natural sources, namely, by extraction from the plant Cannabis sativa L. In some embodiments, the cannabinoids may be derived from natural sources and may carry impurities such as any fatty acids such as linoleic acid and a-linoleic acid, which are natural components of hemp oil or cannabis oil, β-caryophyllene, myrcene, and β-sitosterol. In hemp oil or cannabis oil, cannabinoid content may be by weight up to 90%, up to 80%, up to 75%, up to 70%, up to 60%, 50%, up to 45%, up to 40%, up to 35%, up to 30%, up to 25%, up to 20%, up to 15%, up to 10%, or up to 5%.

In some embodiments, the cannabinoids include prodrugs of cannabinoids. As used herein “prodrug” refers to a compound that undergoes a chemical conversion, through a metabolic process or otherwise within the body of the mammal receiving the compound into its active form that has therapeutic effects. In some embodiments, products of cannabinoids include those described in U.S. Pat. No. 9,533,942, the disclosure of which is incorporated by reference herein in its entirety. Additional embodiments of cannabidiol prodrugs contemplated by the present disclosure include, but are not limited to, those described in U.S. patent application Ser. No. 12/182,974, published as US 2009-0036523 A1 on Feb. 5, 2009, which is incorporated herein by reference in its entirety.

The preparation of pharmaceutically acceptable cannabinoids useful in the nanoparticle compositions of the present disclosure may be accomplished via any procedure known to those skilled in the art. Generally, in the isolation of THC and other cannabinoid constituents from the natural material (e.g., cannabis), the alcoholic or the petroleum ether or benzene or hexane extract of the plant is separated into neutral and acidic fractions, which are then further purified by repeated column chromatography and/or countercurrent distribution. Various adsorbents have been used in column chromatography, especially silica gel, silicic acid, silicic acid-silver nitrate, florisil, acid washed alumina, and acid washed alumina-silver nitrate. U.S. Pat. Nos. 6,365,416 and 6,730,519 describe improvements wherein Cannabis plant material is extracted with a non-polar organic solvent to provide an extract containing THC and the extract is subjected to fractional distillation under reduced pressure to provide a distillation fraction (distillate) having a high content of THC. The process further comprises subjecting the extract from the plant material to column chromatography prior to fractional distillation. A still further aspect of the process comprises subjecting the distillate from the fractional distillation to column chromatography. Additionally, the process uses high pressure liquid chromatography (HPLC) in the purification of the extract from the plant material. Another method of manufacture for obtaining cannabinoids useful in the present disclosure includes the method described in U.S. Pat. Nos. 6,730,519 and 6,365,416 (both to Elsohly, et al.), both hereby incorporated by reference in its entirety. Therein, a method for the isolation of delta-9-tetrahydrocannibinol (THC) from, Cannabis plant material is described wherein delta-9-THC Acid and THC are separately obtained including the steps of extracting the Cannabis plant material, chelating delta-9-THC acid on alumina solid support from cannabis extracts rich in the acid washing of non-acid components of the extract with organic solvents and eluting of the delta-9-THC acid with strong polar solvents.

In some embodiments, the nanoparticle compositions of the present disclosure may comprise the cannabinoid in an amount ranging from between about 0.01% to about 30% by total weight of the composition. In other embodiments, the nanoparticle compositions of the present disclosure may comprise the cannabinoid in an amount ranging from between about 0.01% to about 15% by total weight of the composition. In yet other embodiments, the nanoparticle compositions of the present disclosure may comprise the cannabinoid in an amount ranging from between about 0.01% to about 10% by total weight of the composition. In yet other embodiments, the nanoparticle compositions of the present disclosure may comprise the cannabinoid in an amount ranging from between about 0.1% to about 15% by total weight of the composition. In yet other embodiments, the nanoparticle compositions of the present disclosure may comprise the cannabinoid in an amount ranging from between about 0.1% to about 10% by total weight of the composition. In yet other embodiments, the nanoparticle compositions of the present disclosure may comprise the cannabinoid in an amount ranging from between about 0.01% to about 5% by total weight of the composition. In yet other embodiments, the nanoparticle compositions of the present disclosure may comprise the cannabinoid in an amount ranging from between about 0.1% to about 5% by total weight of the composition. In yet other embodiments, the nanoparticle compositions of the present disclosure may comprise the cannabinoid in an amount ranging from between about 0.01% to about 2% by total weight of the composition. In yet other embodiments, the nanoparticle compositions of the present disclosure may comprise the cannabinoid in an amount ranging from between about 0.1% to about 2% by total weight of the composition.

In some embodiments, the nanoparticle compositions may comprise the API (e.g the cannabinoid) in any concentration that allows it to achieve a concentration in the range of from about 0.001 mg/kg to about 100 mg/kg. In other embodiments, the nanoparticle compositions may comprise the API in any concentration that allows it to achieve a concentration in the range of from about 0.01 mg/kg to about 100 mg/kg. In other embodiments, the nanoparticle compositions may comprise the API in any concentration that allows it to achieve a concentration in the range of from about 0.01 mg/kg to about 80 mg/kg. In other embodiments, the nanoparticle compositions may comprise the API in any concentration that allows it to achieve a concentration in the range of from about 0.01 mg/kg to about 70 mg/kg. In other embodiments, the nanoparticle compositions may comprise the API in any concentration that allows it to achieve a concentration in the range of from about 0.01 mg/kg to about 60 mg/kg. In other embodiments, the nanoparticle compositions may comprise the API in any concentration that allows it to achieve a concentration in the range of from about 0.01 mg/kg to about 50 mg/kg. In other embodiments, the nanoparticle compositions may comprise the API in any concentration that allows it to achieve a concentration in the range of from about 0.1 mg/kg to about 80 mg/kg. In other embodiments, the nanoparticle compositions may comprise the API in any concentration that allows it to achieve a concentration in the range of from about 0.1 mg/kg to about 70 mg/kg. In other embodiments, the nanoparticle compositions may comprise the API in any concentration that allows it to achieve a concentration in the range of from about 0.1 mg/kg to about 60 mg/kg. In other embodiments, the nanoparticle compositions may comprise the API in any concentration that allows it to achieve a concentration in the range of from about 0.1 mg/kg to about 50 mg/kg.

In some embodiments, an amount of water present in the composition ranges from about 90% to about 98% by total weight of the composition, from about 90% to about 97% by total weight of the composition, from about 90% to about 96% by total weight of the composition, from about 90% to about 95% by total weight of the composition, from about 90% to about 94% by total weight of the composition, from about 90% to about 93% by total weight of the composition, from about 90% to about 92% by total weight of the composition, and from about 90% to about 91% by total weight of the composition.

In some embodiments, the nanoparticle compositions comprise an emulsifier present in an amount ranging from about 0.5% to about 50% by total weight of the composition. In some embodiments, the nanoparticle compositions comprise an emulsifier present in an amount ranging from about 0.5% to about 40% by total weight of the composition. In some embodiments, the nanoparticle compositions comprise an emulsifier present in an amount ranging from about 0.5% to about 30% by total weight of the composition. In some embodiments, the nanoparticle compositions comprise an emulsifier present in an amount ranging from about 0.5% to about 20% by total weight of the composition. In some embodiments, the nanoparticle compositions comprise an emulsifier present in an amount ranging from about 0.5% to about 15% by total weight of the composition. In some embodiments, the nanoparticle compositions comprise an emulsifier present in an amount ranging from about 1.5% to about 9.5% by total weight of the composition, from about 1.5% to about 9% by total weight of the composition, from about 1.5% to about 8.5% by total weight of the composition, from about 1.5% to about 8% by total weight of the composition, from about 1.5% to about 7.5% by total weight of the composition, from about 1.5% to about 7% by total weight of the composition, from about 1.5% to about 6.5% by total weight of the composition, from about 1.5% to about 6% by total weight of the composition, from about 1.5% to about 5.5% by total weight of the composition, from about 1.5% to about 5% by total weight of the composition, from about 1.5% to about 4.5% by total weight of the composition, from about 1.5% to about 4% by total weight of the composition, from about 1.5% to about 3.5% by total weight of the composition, from about 1.5% to about 3% by total weight of the composition, from about 1.5% to about 2.5% by total weight of the composition, or from about 1.5% to about 2% by total weight of the composition.

The emulsifier may be any known to those of ordinary skill in the art. In some embodiments, the emulsifier is a block copolymer containing a polyoxyethylene block, i.e. a block made up of repeating ethylene oxide moieties. A suitable emulsifier of this type is Poloxamer, i.e. a polyoxyethylene-polyoxypropylene block copolymer, such as Poloxamer 188. See the Handbook of Pharmaceutical Excipients, p. 352,2nd Edn. Pharmaceutical Press, London, 1994, Eds, Wade and Weller.

In some embodiments, the emulsifier is a phospho emulsifier. This can be any pharmaceutically acceptable material derived from soybeans or eggs, e.g. soy or egg lecithins. Egg lecithins, such as the material provided by Lipoid (Germany) known as Lipoid E80, which contains both phosphatidylcholine and phosphatidyl ethanoline, are preferred, although other phospholipid materials could be used including phospholipid-polyethylene glycol (PEG) conjugates (PEGylated phospholipids) that have been described for use in liposome systems, e. g. by Litzinger et al, Biochem Biophys Acta, 1190 (1994) 99-107.

Exemplary phospholipids suitable for oral dosage forms include: Phosal® 50 PG; Phosal® 53MCT; Phosal® 75SA, Phospholipon® 80; Phospholipon®80H; Phospholipon®85G; Phospholipon® 90G; Phospholipon® 90H; and Phospholipon® 9ONG. Exemplary phospholipids suitable for dermal dosage forms include: Phosal® 50 PG; Phosal® 50SA; Phosal®53MCT; Phosal® 75SA; Phospholipon® 80; Phospholipon® 80H; Phospholipon® 85G; Phospholipon® 90NG; Phospholipon®90G; Phospholipon® 90H; and Phospholipon® 100H. Exemplary phospholipids suitable for parenteral dosage forms include: Phospholipon®90G; Phospholipong90H; and Phospholipon® 100H. Phosholipids suitable for pulmonary drug formulations include: Phospholipon® 90G; Phospholipon® 9.0H and Phospholipon®.

In some embodiments, the emulsifier may comprise a polysaccharide. The polysaccharide may be linear or branched, sulfated or unsulfated. In some embodiments, the composition comprises one or more linear sulfated polysaccharide known as “carrageenan”.

In some embodiments, the emulsifier is a carrageenan, for example one or more of a lambda-carrageenan, kappa-carrageenan, iota-carrageenan and any mixture of the carrageenans. The carrageenans are a family of linear sulfated polysaccharides that are extracted from edible seaweed and widely used in the food industry. The USPNF 23 describes carrageenan as hydrocolloid obtained by extraction and purification with water or aqueous alkali from few members of the class Rhodophyceae (red seaweed). It consists mainly of potassium, sodium, calcium magnesium and ammonium sulfate esters of galactose and 3,6-anhydrogalactose copolymers. These hexoses are alternatively linked at the α-1,3 and (β-1,4 sites in the polymer.

Carrageenans are divided into three families according to the position of sulfate groups and the presence of anhydrogalactose. Lambda-carrageenan (λ-carrageenan) is a nongelling polymer containing about 35% ester sulfate by weight and no 3,6-anhydrogalactose. Iota-carrageenan (t-carrageenan) is a gelling polymer containing about 32% ester sulfate by weight and approximately 30% 3,6-anhydrogalactose. Kappa carrageenan (κ-carrageenan) is a strongly gelling polymer which has a helical tertiary structure that allows gelling. It contains 25% ester sulfate by weight and approximately 34% 3,6-anhydrogalactose. Among the three carrageenans, λ-carrageenan is the only nongelling polymer.

In some embodiments, the emulsifier is selected from the group consisting of a monoglyceride, a diglyceride, and any mixture thereof. The term “monoglyceride” refers to a molecule with one glycerol moiety covalently bonded to a fatty acid chain via an ester bond. The term “diglyceride” refers to a molecule with one glycerol moiety covalently bonded to two fatty acid chains via ester bonds. In some embodiments, the emulsifier includes a mixture of monoglycerides and diglycerides. In some embodiments, the emulsifier includes a mixture of monoglycerides and diglycerides and a carrageenan.

In some embodiments, the emulsifier is a lecithin (which is a generic term to designate any group of fatty substances occurring in animal and plant tissues that are composed of phosphoric acid, choline, fatty acids, glycerol, glycolipids, triglycerides, and phospholipids (e.g., phosphatidylcholine, phosphatidylethanolamine, and phosphatidylinositol)). In some embodiments, triglycerides include vegetable oils, fish oils, animal fats, hydrogenated vegetable oils, partially hydrogenated vegetable oils, medium and long-chain triglycerides, and structured triglycerides. In some embodiments, the emulsifier may be an oil, including vegetable oils such as soybean oil, olive oil, cotton seed oil, peanut oil, sesame oil and castor oil, with sesame oil and castor oil being preferred.

In some embodiments, the emulsifier is a fatty acid ester of polyoxyethylene sorbitan (e.g. Polysorbate® 80, Polysorbate® 20, etc.).

The compositions of the present disclosure may further comprise one or more pharmaceutically acceptable excipients selected from a diluent, a binder, a lubricant, a disintegrant, a flavoring agent, taste-masking agent, a coloring agent, pH modifiers, a stabilizer, absorption enhancers, viscosity modifiers, film forming polymers, bulking agents, a surfactant, a glidant, a plasticizer, a preservative, essential oil and a sweetener. The phrase “pharmaceutically acceptable” refers to molecular entities and compositions that do not produce adverse, allergic, or other untoward reactions when administered to an animal or a human. A person skilled in the art will be able to select the best excipient or mixture of excipients for the desired formulation. Each excipient may fall within one or more classifications.

The amount of each of these components which may be used will be optimized for each formulation, in order to obtain a stable product (dosage form) having the desired shelf-life. Generally speaking, in embodiments in which these components are included, suitable formulations may include from about 0.001% to about 20% w/w of a pharmaceutically acceptable excipient.

Absorption enhancers for use in accordance with certain embodiments of the present disclosure include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like. In certain preferred embodiments, the absorption enhancer is triacetin. In certain embodiments wherein an absorption enhancer is included in the formulation, the absorption enhancer is included in an amount of from about 0.001% to about 10% by weight of the formulation, preferably in an amount of about 0.01% to about 5% by weight of the formulation.

A diluent may be selected from, for example, calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol.

A binder may be selected from, for example, acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol.

A suitable filler may be selected from, for example, starch derivatives, such as corn starch, potato starch or rice starch, polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

A disintegrant may be selected from, for example, alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, sodium starch glycolate, starch, pregelatinized starch, low-substituted hydroxypropyl cellulose.

A glidant may be selected from, for example, calcium silicate, powdered cellulose, starch, talc, colloidal silicon dioxide.

A lubricant may be selected from, for example, magnesium stearate, stearic acid, sodium stearyl fumarate, magnesium lauryl sulphate, talc, polyethylene glycol, and glyceryl behenate.

A suitable essential oil may be selected from Bergamot oil (extracted from Citrus aurantium L. subsp. bergamia Wright et Am.); Ylang ylang oil (extracted from Cananga odorata Hook. f. and Thoms.); Jasmine essential oil (extracted from Jasminum officinale L.). In one embodiment, a mixture of essential oils comprises equal portions totaling about 0.01% to about 1% w/w, preferably about 0.1% w/w of the total composition. Other essential oils are possible.

A suitable sweetener may be selected from sugars such as sucrose, lactose and glucose; cyclamate and salts thereof saccharin and salts thereof and aspartame.

A flavoring agent may be selected from natural or synthetic flavors such as, for example, strawberry flavor, wild cherry flavor, green apple flavor, spearmint flavor and peppermint flavor.

Any surfactant may be one of an anionic surfactant, a cationic surfactant, a non-ionic surfactant, or mixtures thereof In some embodiments, an appropriate surfactant is selected such that (i) when combined with the other reagent components, it allows for the desired surface tension to be achieved; (ii) does not denature proteins or other reagent components; and/or (iii) it provides a low foam height.

Anionic surfactants are generally based upon sulfates, sulfonates, phosphates, or carboxylates and contain a water-soluble cation. A representative formula of a sulfonate is R—SO3M where R is a hydrocarbon group of from about 5 to 22 carbon atoms which may be linked through an alkoxy or oxyalkoxy to the sulfonate functionality and M is a water-soluble cation such as an alkali metal. Anionic surfactants include alkyl ether sulfates, alkyl sulfates and sulfonates, alkyl carboxylates, alkyl phenyl ether sulfates, sodium salts of alkyl poly(oxyethylene) sulfonates, sodium salts of alkyl benzyl sulfonate, such as sodium salts of dodecylbenzyl sulfonate and sodium lauryl ether sulfate. Anionic surfactants also include anionic phosphate esters.

In some embodiments, the surfactants include, but are not limited to polyoxyethylene alkyl ether, wherein the alkyl is (CH₂)_{M} and the oxyethylene is (C₂H₄O)N, wherein M is an integer from 5 to 16, from 8 to 14, or from 10 to 12 and N is an integer from 10 to 40, from 15 to 30, or from 20 to 28. In one embodiment, the surfactant is polyoxyethylene lauryl ether having a formula (C₂H₄O)₂₃C₁₂H₂₅OH. In another embodiment, the surfactant is a polyoxyethylene (20) sorbitan monoalkylate, the monoalkylate comprising between 8 and 14 carbons. In another embodiment, the surfactant is a linear secondary alcohol polyoxyethylene having a formula C₁₂₋₁₄H₂₅₋₂₉O(CH₂CH₂O]ₓ, wherein x equals an integer between 2 and 12. In yet another embodiment, the surfactant is a polyoxyethylene octyl phenyl ether. Exemplary surfactants are sold under the names: Brij® 35, TWEEN®, Tergitol™, Triton™, Ecosurf™, Dowfax™ polysorbate 80™, BigCHAP, Deoxy BigCHAP, IGEPAL®, Saponin, Thesit®, Nonidet®, Pluronic F-68, digitonin, deoxycholate, and the like. Particular disclosed working embodiments concern using surfactants selected from Brij® 35, TWEEN®, Tergitol™, Triton™.

In some embodiments, the surfactant is polysorbate 80 (i.e. Polyoxyethylene (20) sorbitan monooleate), having the structure below:

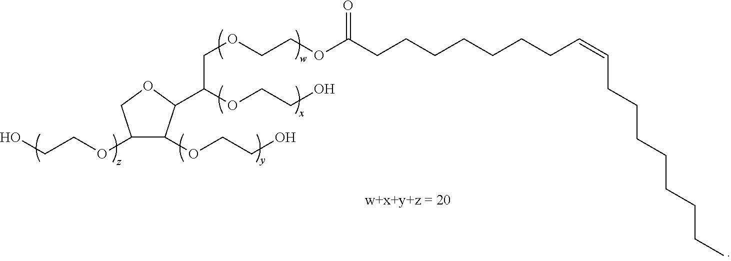

Cationic surfactants useful in compositions of the present disclosure contain amino or quaternary ammonium moieties. Cationic surfactants among those useful herein are disclosed in the following documents: M.C. Publishing Co., McCutcheon's, Detergents & Emulsifiers, (North American edition 1979); Schwartz, et al.; Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr., issued Jun. 7, 1983.

Among the quaternary ammonium-containing cationic surfactant materials useful herein are those of the general formula:

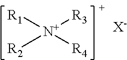

wherein R1-R4 are independently an aliphatic group of from about 1 to about 22 carbon atoms or an aromatic, alkoxy, polyoxyalkylene, alkylamido, hydroxyalkyl, aryl or alkylaryl group having from about 1 to about 22 carbon atoms; and X is a salt-forming anion such as those selected from halogen, (e.g. chloride, bromide), acetate, citrate, lactate, glycolate, phosphate nitrate, sulfate, and alkylsulfate radicals. The aliphatic groups may contain, in addition to carbon and hydrogen atoms, ether linkages, and other groups such as amino groups. The longer chain aliphatic groups, e.g., those of about 12 carbons, or higher, can be saturated or unsaturated. Especially preferred are mono-long chain (e.g., mono C₁₂ to C₂₂, preferably C₁₂ to C₁₈, more preferably C₁₆, aliphatic, preferably alkyl), di-short chain (e.g., C₁ to C₃ alkyl, preferably C₁ to C₂ alkyl) quaternary ammonium salts.

Salts of primary, secondary and tertiary fatty amines are also suitable cationic surfactant materials. The alkyl groups of such amines preferably have from about 12 to about 22 carbon atoms, and may be substituted or unsubstituted. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (with 5 moles of ethylene oxide) stearylamine, dihydroxy ethyl stearylamine, and arachidylbehenylamine. Suitable amine salts include the halogen, acetate, phosphate, nitrate, citrate, lactate, and alkyl sulfate salts. Such salts include stearylamine hydrochloride, soyamine chloride, stearylamine formate, N-tallowpropane diamine dichloride, stearamidopropyl dimethylamine citrate, cetyl trimethyl ammonium chloride and dicetyl diammonium chloride. Preferred for use in the compositions herein are cetyl trimethyl ammonium chloride, stearyl trimethyl ammonium chloride, tetradecyltrimethly ammonium chloride, dicetyldimethyl ammonium chloride, dicocodimethyl ammonium chloride and mixtures thereof. More preferred is cetyl trimethyl ammonium chloride.

The compositions of the disclosure may also include various non-ionic surfactants. Among the suitable nonionic surfactants are condensation products of C₈-C₃₀ alcohols with sugar or starch polymers. These compounds can be represented by the formula (S)ₙ—O—R, wherein S is a sugar moiety such as glucose, fructose, mannose, and galactose; n is an integer of from about 1 to about 1000, and R is C₈-C₃₀ alkyl. Examples of suitable C₈-C₃₀ alcohols from which the R group may be derived include decyl alcohol, cetyl alcohol, stearyl alcohol, lauryl alcohol, myristyl alcohol, oleyl alcohol, and the like. Specific examples of these surfactants include decyl polyglucoside and lauryl polyglucoside.

Other suitable nonionic surfactants include the condensation products of alkylene oxides with fatty acids (i.e., alkylene oxide esters of fatty acids). These materials have the general formula RCO(X)ₙ OH, wherein R is a C₁₀-C₃₀ alkyl, X is —OCH₂CH₂— (derived from ethylene oxide) or —OCH₂CHCH₃— (derived from propylene oxide), and n is an integer from about 1 to about 200.

Yet other suitable nonionic surfactants are the condensation products of alkylene oxides with fatty acids (i.e., alkylene oxide diesters of fatty acids) having the formula RCO(X)ₙOOCR, wherein R is a C₁₀-C₃₀ alkyl, X is —OCH₂CH₂— (derived from ethylene oxide) or —OCH₂CHCH₃— (derived from propylene oxide), and n is an integer from about 1 to about 200. Yet other nonionic surfactants are the condensation products of alkylene oxides with fatty alcohols (i.e., alkylene oxide ethers of fatty alcohols) having the general formula R(X)ₙOR′, wherein R is C₁₀-C₃₀ alkyl, n is an integer from about 1 to about 200, and R′ is H or a C₁₀-C₃₀ alkyl.

Still other nonionic surfactants are the compounds having the formula RCO(X)ₙOR′ wherein R and R′ are C₁₀-C₃₀ alkyl, X is —OCH₂CH₂— (derived from ethylene oxide) or —OCH₂CHCH₃— (derived from propylene oxide), and n is an integer from about 1 to about 200. Examples of alkylene oxide-derived nonionic surfactants include ceteth-1, ceteth-2, ceteth-6, ceteth-10, ceteth-12, ceteraeth-2, ceteareth6, ceteareth-10, ceteareth-12, steareth-1, steareth-2, stearteth-6, steareth-10, steareth-12, PEG-2 stearate, PEG4 stearate, PEG6 stearate, PEG-10 stearate, PEG-12 stearate, PEG-20 glyceryl stearate, PEG-80 glyceryl tallowate, PPG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof. Still other useful nonionic surfactants include polyhydroxy fatty acid amides disclosed, for example, in U.S. Pat. Nos. 2,965,576, 2,703,798, and 1,985,424, which are incorporated herein by reference.

Exemplary surfactants include Tomadol 1200 (Air Products), Tomadol 900 (Air Products), Tomadol 91-8 (Air Products), Tomadol 1-9 (Air Products), Tergitol 15-S-9 (Sigma), Tergitol 15-S-12 (Sigma), Masurf NRW-N (Pilot Chemical), Bio-Soft N91-6 (Stepan), and Brij-35 (Polyethylene glycol dodecyl ether) (Sigma).

The pharmaceutical compositions may comprise penetration enhancers to enhance their delivery. Penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di-glycerides and physiologically acceptable salts thereof. The compositions may further include chelating agents such as, for example, ethylenediaminetetraacetic acid (EDTA), citric acid, salicylates (e.g. sodium salycilate, 5-methoxysalicylate, homovanilate). The expression vectors disclosed herein (or combinations thereof) may be delivered combined with minicells or nanoparticles.

Routes of Administration and Dosage Forms

Administration to a subject of the pharmaceutical compositions according to the present disclosure may be via any common route so long as the target tissue is available via that route.

Liquid dosage forms for oral administration include, but are not limited to, pharmaceutically acceptable emulsions, solutions, suspensions, syrups and elixirs. In addition to the cannabinoid, the liquid dosage forms can contain inert diluents commonly used in the art. For instance, liquid formulations can contain water, alcohol, polyethylene glycol ethers, or any other pharmaceutically acceptable solvents. Solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof may also be present in the inventive compositions. Additionally, oral compositions can include adjuvants such as wetting agents, emulsifying and suspending agents, sweetening, flavoring, and perfuming agents. When formulated as a suspension, the inventive compositions contain the cannabinoid extract and suspending agents, for example, ethoxylated isostearyl alcohols, polyoxyethylene sorbitol, sorbitan esters, microcrystalline cellulose, aluminum metahydroxide, bentonite, agar-agar, tragacanth, and mixtures thereof.

A dietary composition or supplement according to the present disclosure is any ingestible preparation that contains the cannabinoid compositions of the present disclosure mixed with a food products or compositions. The food product can be dried, cooked, boiled, lyophilized or baked. A food composition or food product can comprise a bakery product, including but not limited to bread, pastries, brownies, cakes, pies, donuts, crackers, and muffins. A food composition or food product can comprise a dairy product, including but not limited to milk, fermented milk, curd, whey, yogurt, cream, cheese, butter, clarified butter, ghee, and ice cream. A food composition or food product can comprise a nut butter or seed butter, including but not limited to peanut butter, almond butter, cashew butter, hazelnut butter, macadamia nut butter, pecan butter, pistachio butter, walnut butter, pumpkin seed butter, sesame seed butter, soybean butter, and sunflower seed butter. A food composition or food product can comprise an oil (e.g., a cooking oil), including but not limited to olive oil, coconut oil, vegetable oil, canola oil, corn oil, peanut oil, sunflower seed oil, almond oil, avocado oil, rice bran oil, cottonseed oil, flaxseed oil, linseed oil, grape seed oil, hemp oil, mustard oil, macadamia oil, palm oil, tea seed oil, walnut oil, margarine, lard, butter, clarified butter, ghee, or tallow. A food composition or food product can comprise sports food products such as energy gels, sports drinks, energy powders, energy bars, energy shots, protein powders, and protein drinks (e.g., protein shakes). A food composition or food product can comprise a beverage, including but not limited to water, electrolyte drinks, soda, coconut water, tea (e.g., Jun tea, black tea, green tea, white tea, herbal tea), coffee, a soft drink, an alcoholic beverage (e.g., cocktail, liquor, spirits, beer, wine, malt beverage), water, juice (e.g., apple juice, orange juice, tomato juice, vegetable juice, cranberry juice), a sports drink, electrolyte-enriched water, vitamin-enhanced water, a hangover-recovery drink, milk (e.g., dairy-based milk, coconut milk, almond milk, soy milk, hemp milk, rice milk, oat milk, cashew milk, hazelnut milk), and yogurt.

The dietary composition or supplement may also comprise one or more additional ingredients, including but not limited to mushrooms or mushroom derivative products (e.g., reishi mushroom, chaga mushroom, maitake mushroom, oyster mushroom, cordyceps), maca (Lepidium meyenii), he sho wu (also he show wu or shou wu chih), superfoods or superfood derivative products (e.g., blueberries, acai berries, inca berries, goji berries, camucamu, coconut, lucuma, kale, cacao (e.g., cacao powder, cacao butter), sacha inchi, chia, flax, hemp, amaranth, quinoa, moringa oleifera), and combinations thereof.

The nanoparticle compositions disclosed herein may be formulated into candies, lollipops, lozenges, etc.

Solid dosage forms suitable for oral administration include, capsules, tablets, pills, powders, and granules. In such solid dosage forms, the cannabinoid compositions may be mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For capsules, tablets and pills, the dosage form can also comprise buffering agents.

Dosage forms for topical administration include, but are not limited to, ointments, creams, emulsions, lotions, gels, sunscreens and agents that favor penetration within the epidermis. Various additives, known to those skilled in the art, may be included in the topical formulations of the present disclosure. Examples of additives include, but are not limited to, solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers. Examples of moisturizers include jojoba oil and evening primrose oil. Suitable skin permeation enhancers are well known in the art and include lower alkanols, such as methanol ethanol and 2-propanol; alkyl methyl sulfoxides such as dimethylsulfoxide (DMSO), decylmethylsulfoxide (C10 MSO) and tetradecylmethyl sulfoxide; pyrrolidones, urea; N,N-diethyl-m-toluamide; C₂-C₆ alkanediols; dimethyl formamide (DMF), N,N-dimethylacetamide (DMA) and tetrahydrofurfuryl alcohol. Examples of solubilizers include, but are not limited to, hydrophilic ethers such as diethylene glycol monoethyl ether (ethoxydiglycol, available commercially as Transcutol®) and diethylene glycol monoethyl ether oleate (available commercially as Softcutol®); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol®); alkyl methyl sulfoxides, such as DMSO; pyrrolidones, DMA, and mixtures thereof.

Cannabinoids can be present in any dosage form in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in any dosage form in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in any dosage form in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in any dosage form in a quantity of from about 50 to about 150 milligrams. Cannabinoids can be present in any dosage form in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be present in any dosage form in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%), 35%), 40%), 45%), or 50% by weight of the product. Cannabinoids can be present in any dosage form in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

Methods of Treatment

The nanoparticle compositions, or any dosage form comprising the nanoparticle compositions, may be used in the treatment of any disorder for which a cannabinoid has therapeutic properties. Non-limiting examples of such disorders include AIDS, cancer, and malignant tumors, which are often accompanied with a lack of appetite, nausea, and vomiting, chronic pain (especially neuropathic pain), spasticity (e.g., in multiple sclerosis and spinal cord injury), dystonia, intractable pediatric epilepsy. Other disorders treatable with the formulation according to the present disclosure include oxidation-associated disease such as myocardial infarction, stroke (motor or sensory abnormalities and cerebral infarct, or a neurovascular thromboembolic event.

The composition may be administered in combination with other therapeutic drugs. The amount and manner of treatment comprising administration of the cannabinoid composition according to the disclosure is determined by a medical professional.

The compositions may also be prepared for recreational use or use as a supplement.

One of ordinary skill will appreciate that effective amounts of the agents in the compositions used in the methods of the present disclosure can be determined empirically. It will be understood that, when administered to a human patient, the total daily usage of the composition of the present disclosure will be decided by the attending physician within the scope of sound medical judgment. The specific therapeutically effective dose level for any patient will depend upon a variety of factors: the type and degree of the response to be achieved; the activity of the specific composition employed; the age, body weight, general health, sex and diet of the patient; the duration of the treatment; drugs used in combination or coincidental with the method of the present disclosure; and like factors well known in the medical arts.

Any of the compositions described above can be provided in a unit dosage form. A unit dosage is an amount of a compound, such as a cannabinoid compound delivered alone or in combination with other components, which is to be administered to a subject at or about one time point. Other components which can be included with a unit dosage include but are not limited to cosmetics, food carriers, food bars, baked goods, dairy products, oils, beverages, solid dosages (e.g., tablets), or liquid dosages. A unit dosage of a cannabinoid compound can be about 1, 1.25, 1.5, 1.75, 2, 2.5, 3, 3.5, 4, 4.5, 5, 5.5, 6, 7, 8, 9, 10, 15, 20, 25, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at least about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at most about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage can be an hourly dosage. A unit dosage can be a daily dosage. A unit dosage can provide about 1/24, 1/12, ⅛, ⅙, ¼, ⅓, ½, or all of a daily dosage of one or more cannabinoids for a subject. A unit dosage can take the form of a tablet, gel, liquid, food product, food bar, container of liquid of defined volume, or other forms described herein, packaged for one-time consumption or administration.

In some embodiments, the compositions disclosed herein may be administered in a combination therapy, i.e., either simultaneously in single or separate dosage forms or in separate dosage forms within hours or days of each other. Examples of compounds/drugs used in such combination therapies include without limitation, chemotherapeutic agents, immunosuppressive agents, immunostimulatory, anti-pyretic, cytokines, opioids, cytokines, cytotoxic agents, nucleolytic compounds, radioactive isotopes, receptors, pro-drug activating enzymes, which may be naturally occurring or produced by recombinant methods, anti-inflammatory agents, antibiotics, protease inhibitors, growth factors, osteo-inductive factors and the like.

Stability and Pharmacokinetics

The compositions described herein provide several advantages, including but not limited to, increased shelf stability, increased bioavailability, and increased bioactivity.

In some embodiments, the cannabinoid compositions of the present disclosure do not degrade to an unacceptable extent such that the final product (cannabinoid dosage form) has a shelf-life of at least about 2 years. As previously mentioned, this means that the active ingredient within the dosage form remains within 90-110% of its initial amount in the dosage form during the desired (e.g., labeled) shelf-life of the dosage form (e.g., a minimum of 2 years after the date of manufacture of the dosage form).

The compositions described herein can have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some cases, the compositions described herein can have a shelf half-life of at least about 1, 2, 3, 4, or 5 years. The nanoparticle compositions described herein may be characterized by a cannabinoid degradation rate at an ambient temperature of at least 20° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of a non-encapsulated cannabinoid composition.

The nanoparticles compositions described herein may be characterized by a plasma half-life in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, or 5.0 times that of a non-nanoparticle cannabinoid composition. Plasma half-life of a composition can be determined experimentally by administering the composition to a subject, taking plasma samples from a subject at multiple time points, and measuring the concentration of the compound or compounds of interest in those plasma samples. The concentration of the compound or compounds of interest will reach a peak value in the plasma, then fall as the compound or compounds are metabolized, degraded, or cleared from the blood stream. The plasma half-life is the time for the plasma concentration value to be halved.

Generation of Nanoparticles

In general, the nanoparticle compositions of the present disclosure may formed by any methods known to those of ordinary skill in the art. In some embodiments, a solution comprising the cannabinoid, water, and the emulsifier provided to a microfluidic droplet device. In some embodiments, the fluid is flowed through the microfluidic device at a pressure of at least 10,000 psi. In some embodiments, the fluid is flowed through the microfluidic device at a pressure of at least 15,000 psi. In some embodiments, the fluid is flowed through the microfluidic device at a pressure of at least 20,000 psi.

In some embodiments, the microfluidizer has a well-defined axially-varying microchannel geometry through which macroemulsions are pumped. In the narrow throats spaced along the length of the microchannel, the drops experience high shear rates and break to form smaller droplets. In some embodiments, multiple passes through the microfluidizer are used.

### EXAMPLE 1

To a 500 mL beaker was added 250 mL water and a magnetic stir bar. The beaker was placed onto a stir plate and set to 200 rpm. Polysorbate 80, 2.5 g, was added to the water and allowed to stir until it fully went into solution. Into the water was added 3.25 g of cannabis oil. The oil was allowed to stir until it went into solution. The solution was poured into a microfluidizer. The microfluider was set to 20,000 psi and the fluid was processed through the system and then collected. The collected material was processed through the system two additional passes.

The final solution was analyzed for particle size and distribution using a Microtrac Nano-flex dynamic light scattering instrument. The cannabinoid content was measured by a contract cannabis analytical lab as detailed in the attached particle size analysis document.

### EXAMPLE 2

High Performance Liquid Chromatography (HPLC) with Ultraviolet Detection (UV) was used to determine the concentration of five different cannabinoids and to calculate cannabinoid potency. This procedure was applicable for the preparation, analysis, and quantification of cannabis flower extracts, cannabis concentrates, and cannabis edibles.

Extracts are prepared using approximately 0.2 g of sample in methanol or water/acetonitrile extraction solutions. The sample extracts were then filtered into 2 ml autosampler vials using a 0.45 um syringe filter and placed on the instrument. A 5 uL aliquot was then injected onto the analytical column and the five cannabinoids were separated on the HPLC column using an acetonitrile/water/formic acid mobile phase. The analytes were detected using a UV detector at 220 nm. The potency was then determined from the dry-weight corrected concentration of each analyte, and was reported as a percent of the total cannabinoid content.

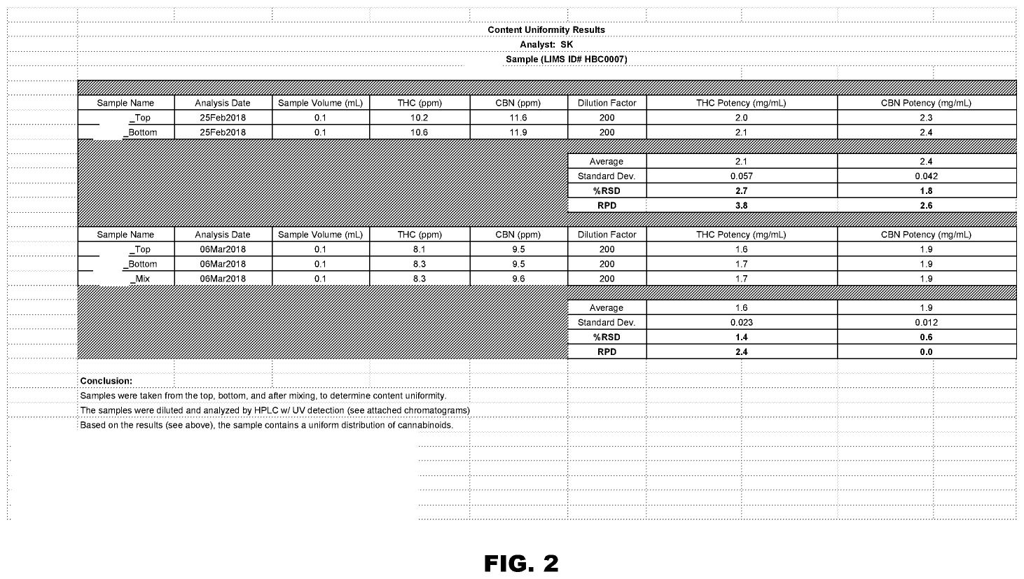

All analyte results were generated in ppm but are reported in % (percent weight). The results are illustrated in FIG. 2. Based on the results, the samples tested contained a uniform distribution of cannabinoids.

All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Although the present disclosure has been described with reference to several illustrative embodiments, it should be understood that many other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A nanoparticle composition comprising an emulsifier and a cannabinoid, wherein nanoparticles within the nanoparticle composition have an average particle size ranging from between about 5 nm to about 50 nm, and wherein the nanoparticles are prepared according to the process of flowing a fluid comprising water, the emulsifier, and the cannabinoid through a microfluidizer at a pressure of at least about 20,000 psi.

2. An solid dosage form comprising nanoparticles having an average particle size ranging from between about 5 nm to about 50 nm, wherein the nanoparticles comprise a cannabinoid and an excipient comprising a block copolymer having a polyethyleneoxide block; wherein the solid dosage form is a candy, a lollipop, or a lozenge; and wherein the nanoparticles are prepared according to the process of flowing a fluid comprising water, the emulsifier, and the cannabinoid through a microfluidizer at a pressure of at least about 20,000 psi.

3. An liquid dosage form comprising nanoparticles having an average particle size ranging from between about 5 nm to about 50 nm, wherein the nanoparticles comprise a cannabinoid and an excipient comprising a block copolymer having a polyethyleneoxide block; wherein the liquid dosage form is a beverage, wherein the beverage is selected from the group consisting of, coconut water, juice, sports drinks, electrolyte-enriched water, vitamin-enhanced water, hangover-recovery drinks, coconut milk, almond milk, soy milk, hemp milk, rice milk, oat milk, cashew milk, and hazelnut milk; and wherein the nanoparticles are prepared according to the process of flowing a fluid comprising water, the emulsifier, and the cannabinoid through a microfluidizer at a pressure of at least about 20,000 psi.
